(19) United States
(12) Patent Application Publication
(10) Pub. No.: US 2023/0159978 A1
(43) Pub. Date: May 25, 2023

(54) Screening of Engineered Biocatalysts for Oxyfunctionalization of Olefins

(71) Applicant: ExxonMobil Technology and Engineering Company, Annandale, NJ (US)

(72) Inventors: Xiaozhou Zhang, Bridgewater, NJ (US); Mohor Chatterjee, Annandale, NJ (US)

(21) Appl. No.: 17/930,462

(22) Filed: Sep. 8, 2022

Related U.S. Application Data
(60) Provisional application No. 63/264,394, filed on Nov. 22, 2021.

(51) Int. Cl. C12Q 1/28; C12P 7/04; C12P 7/24; C12P 3/00; C12N 9/04; C12N 9/88

(52) U.S. Cl. CPC C12Q 1/28; C12P 7/04; C12P 7/24; C12P 3/00; C12N 9/0006; C12Y 101/0302; C12N 9/88; C12Y 402/01053

(57) ABSTRACT

A variety of methods and systems for screening biocatalysts are disclosed, including, in one embodiment, a screening method for identifying engineered biocatalysts, including reacting an olefin with water in the presence of an engineered biocatalyst to produce at least a fatty alcohol having from 4 carbons to 24 carbons; reacting at least a portion of the fatty alcohol with oxygen in the present of a fatty alcohol oxidase to produce a fatty aldehyde and hydrogen peroxide, the fatty aldehyde having from 4 carbons to 24 carbons; and measuring activity of the engineered biocatalyst.

### CROSS-REFERENCE TO RELATED APPLICATIONS

This application claims the benefit of U.S. Provisional Application No. 63/264,394, filed on Nov. 22, 2021, the entire contents of which are incorporated herein by reference.

### FIELD

This application relates to olefin production and, more particularly, embodiments relate to methods and system for screening biocatalysts to identify engineered enzymes or microbes that can oxyfunctionalize olefins with improved performance.

### BACKGROUND

Olefines are widely used as starting raw materials in preparing a wide variety of fuels and chemicals. Fatty alcohols are example of a chemical that can made from olefins and are an important precursor for making ethers, which can be used as fuel and manufacture of high-value chemicals. Current commercial process for production of fatty alcohols includes the catalyst-driven oxidation of α-olefins. However, the current commercial catalysts require harsh conditions with no stereoselectivity and low yields towards long-chain olefins.

### SUMMARY

Disclosed herein is an example screening method for identifying engineered biocatalysts, including reacting an olefin with water in the presence of an engineered biocatalyst to produce at least a fatty alcohol having from 4 carbons to 24 carbons; reacting at least a portion of the fatty alcohol with oxygen in the present of a fatty alcohol oxidase to produce a fatty aldehyde and hydrogen peroxide, the fatty aldehyde having from 4 carbons to 24 carbons; and measuring activity of the engineered biocatalyst.

Further disclosed herein is an example screening system for identifying biocatalysts, including: one or more recombinant plasmids configured to express an engineered biocatalyst that oxyfunctionalizes olefins and a fatty alcohol oxidase that oxidizes fatty alcohols; and a hydrogen peroxide assay.

These and other features and attributes of the disclosed methods and systems of the present disclosure and their advantageous applications and/or uses will be apparent from the detailed description which follows.

### BRIEF DESCRIPTION OF THE DRAWINGS

To assist those of ordinary skill in the relevant art in making and using the subject matter hereof, reference is made to the appended drawings, wherein:

FIG. 1 is an illustrative depiction of a flow chart for screening of engineered biocatalyst in accordance with certain embodiments of the present disclosure.

FIG. 2 is a schematic diagram of a screening system for engineered biocatalysts based on intracellular co-expression of an engineered fatty acid hydratase and a fatty alcohol oxidase.

FIG. 3 is a schematic diagram of a screening system for engineered biocatalysts based on co-expression and secretion of engineered fatty acid hydratase and a fatty alcohol oxidase into the periplasmic space.

### DETAILED DESCRIPTION

Disclosed herein are methods and systems for screening biocatalysts to identify engineered enzymes or microbes that can oxyfunctionalize olefins with improved performance. In accordance with present embodiments, a screening system for engineered biocatalysts is based on two coupled enzymatic reactions with a hydrogen peroxide assay. Example embodiments include a first enzymatic reaction that is the catalyzed reaction of an olefin with water in the presence of an engineered biocatalyst to produce a fatty alcohol. Example embodiments include a second enzymatic reaction that is the catalyzed reaction of the fatty alcohol with oxygen in the presence of a fatty alcohol oxidase to produce a fatty aldehyde and hydrogen peroxide. Example embodiments then use a hydrogen peroxide assay to indirectly measure the activity of the engineered biocatalyst. While the hydrogen peroxide assay is typically performed extracellular, the coupled enzymatic reactions can be designed as whole cell or cell-free systems.

Oxyfunctionalization of olefins includes the addition of water to form fatty alcohols. However, the C—H bonds in olefins are strong and, thus, their activation can be challenging. In accordance with present embodiments, biocatalysts, such as hydratases, are used to catalyze the oxifunctionalization of olefins. However, a number of biocatalysts do not perform at a rate that make their use economically. Thus, example embodiments include alteration biocatalyst, such as hydratases, to form engineered biocatalyst with changed properties such that their use is economically viable.

A variety of different engineered biocatalysts can be readily prepared but screening the biocatalysts to determine their relative performance can be challenging. For example, conventional screening systems for biocatalysts use cell lysis for cell disruption and extraction of cellular contents that require expensive and complex laboratory equipment. In addition, screening systems based on cell lysis are not conducive to high throughput so can delay the iterative cycle needed for enzyme (biocatalyst) selection. Advantageously, present embodiments provide a screening system based on two coupled enzymatic reactions with a hydrogen peroxide assay. In some embodiments, the screening system can be implemented with single whole cell with co-expression of multiple enzymes. The screening system does not require cell lysis, thus includes less steps for development of the hydrogen peroxide assay. In addition, because the adaption of the colorimetric assay for enzyme activity assay, example embodiments of the screening system can be implemented on a high-throughput liquid handler platform, thus reducing the iterative cycle to engineer an enzyme with the desired catalytic activity.

### Definitions

As used herein, the term “engineered biocatalyst” refers to an enzyme or other microbe that can function as a biocatalyst that has been altered through human manipulation. For example, engineered biocatalyst include enzymes or microbes with modified structures, modified functions, and/or modified catalytic activity. Non-limiting examples of human manipulations for biocatalyst alteration include both chemical modification, mutagenesis, and directed enzyme evolution. A specific example of an engineered biocatalyst includes an engineered hydratase.

As used herein, the term “fatty alcohol” refers to an aliphatic, long-chain alcohol having a carbon chain length ranging from 4 carbons to 26 carbons. Specific examples of fatty alcohols include lauryl, stearyl, and oleyl alcohols. In some embodiments, fatty alcohols are saturated or unsaturated. In some embodiments, fatty alcohols are straight chain or branched.

As used herein, the term “fatty aldehyde” refers to an aliphatic, long-chain aldehyde having a carbon chain length ranging from 4 carbons to 26 carbons. Specific examples of fatty aldehydes include octanal, nonanal, and dodecanal.

As used herein, the term “recombinant plasmid” refers to a plasmid that has been altered through human manipulation. Plasmids are extrachromosonal DNA within a cell separated from chromosomal DNA that can replicate independently. Advances in biochemistry in recent years have led to the construction of “recombinant” cloning media, in which plasmids are caused to contain exogenous DNA. In particular cases, the recombinant may contain “heterologous” DNA, by which is meant DNA encoding polypeptides that are not normally produced by the organism susceptible to transformation by the recombinant medium. Plasmids are capable of expressing particular enzymes. Thus, plasmids can be altered to express engineered enzymes.

### EXAMPLE EMBODIMENTS

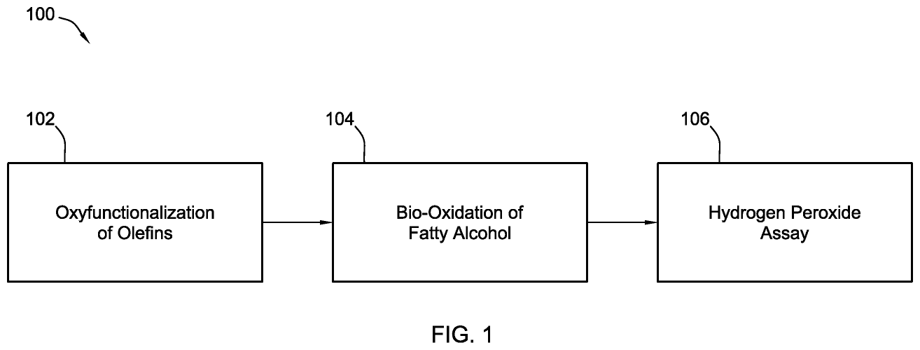

FIG. 1 is a flow chart of a screening method 100 for engineered biocatalyst in accordance with certain embodiments of the present disclosure. The screening method 100 can be used in the evaluation of engineered biocatalyst to identify engineered biocatalyst that can oxyfunctionalize olefins with improved performance. In block 102, the screening method 100 includes the oxyfunctionalization of an olefin in the presence of an engineered biocatalyst to form a fatty alcohol. In block 104, the screening method 100 includes the bio-oxidation of the fatty alcohol in the presence of a fatty alcohol oxidase to form a fatty aldehyde and hydrogen peroxide. In block 106, the screening method 100 includes a hydrogen peroxide assay that determines concentration of the hydrogen peroxide. From this determination, the activity of the engineered biocatalyst for oxyfunctionalization can be indirectly determined.

Block 102 includes the oxyfunctionalization of an olefin in the presence of an engineered biocatalyst to form a fatty alcohol. For example, block 102 includes the catalyzed reaction of an olefin with water in the presence of an engineered biocatalyst (e.g., engineered hydratase) to produce a fatty alcohol. The olefin used in the oxyfunctionalization of block 102 can be any suitable olefin. Examples of suitable olefins include olefins having from 4 carbons to 26 carbons, such as nonene (e.g., non-1-ene) and decene (e.g., dec-1-ene). In some embodiments, the olefins include α-olefins having the double bond at the primary position, thus providing more reactivity. In some embodiments, the olefins include internal olefins. In some embodiments, the olefins are straight chain or branched.

In FIG. 1, the engineered biocatalyst that catalyzes the reaction of the olefin and water in block 102 is expressed from a recombinant plasmid in accordance with one or more embodiments. The recombinant plasmid expresses the engineered enzyme that can catalyze the addition of water to the olefin. In some embodiments, the engineered biocatalyst includes engineered fatty acid hydratases, such as oleic acid hydratase or other hydratases that are active towards unsaturated fatty acids. The recombinants plasmids can be prepared that can express a plurality of variants of the engineered biocatalysts, such as variants of oleic acid hydratases.

Block 104 includes the bio-oxidation of the fatty alcohol in the presence of a fatty alcohol oxidase to form a fatty aldehyde and hydrogen peroxide. For example, block 104 include the catalyzed reaction of the fatty alcohol with oxygen in the presence of a fatty alcohol oxidase to produce a fatty aldehyde and hydrogen peroxide. The fatty alcohol oxidase is expressed from a recombinant plasmid in accordance with one or more embodiments. Suitable fatty alcohol oxidases include enzymes that can oxidize the primary or secondary fatty alcohol to fatty aldehyde with byproduct of H₂O₂.

In some embodiments, the reactions of block 102 and block 104 are whole cell systems with reactions performed inside single whole cells. In some embodiments, the method includes co-expression of the engineered biocatalyst (e.g., engineered enzyme or engineered microbe) and the fatty alcohol oxidase. For example, the screening method 100 includes intracellular co-expression of both the engineered enzyme and the fatty alcohol oxidase into the cytoplasm. The olefin is transported into the cytoplasm where reaction with water is catalyzed by the engineered biocatalyst in accordance with present embodiments. By way of further example, the screening method 100 includes intracellular co-expression of both the engineered biocatalyst and the fatty alcohol oxidase into the cytoplasm followed by secretion of the co-expressed enzymes into the periplasmic space. The olefin is transported across the outer membrane of the cell into the periplasm where reaction with water is catalyzed by the engineered biocatalyst in accordance with present embodiments. While the preceding description describes whole cell techniques, it should be understood that cell-free in vitro coupled enzymatic reactions should also work for screening of biocatalyst. For example, reactions can be performed with cell-free extracts from cells containing the expressed enzymes.

In some embodiments, the method 100 includes a host cell. For example, the recombination plasmid is introduced into a host cell. Examples of suitable host cells include any host cell or organism that is suitable for the production an enzyme of interest. In some embodiments, the host cell is a prokaryotic or a eukaryotic host cell. The host cell may be a host cell that is suitable for culture in liquid or on solid media.

Examples of suitable host cells include microbial host cells such as bacterial or fungal cells. Suitable bacterial host cells include both Gram-positive and Gram-negative bacteria. Examples of suitable bacterial host cells include bacteria from the genera Bacillus, Actinomycetis, Escherichia, Streptomyces as well as lactic acid bacteria such as Lactobacillus, Streptococcus, Lactococcus, Oenococcus, Leuconostoc, Pediococcus, Camobacterium, Propionibacterium, Enterococcus and Bifidobacterium. Particularly preferred are Bacillus subtilis, Bacillus amyloliquefaciens, Bacillus licheniformis, Escherichia coli, Streptomyces coelicolor. Streptomyces clavuligerus, and Lactobacillus plantarum, Lactococcus lactis.

Additional examples of suitable host cells include eukaryotic microorganism such as a yeast or a filamentous fungus. Preferred yeasts as host cells belong to the genera Saccharomyces, Kluyveromyces, Candida, Pichia, Schizosaccharomyces, Hansenula, Kloeckera, Schwarmiomyces, and Yarrowia. Specific examples include Debaromyces host cells, such as Saccharomyces cerevisiae and Kluyveromyces lactis.

Block 106 incudes use of a hydrogen peroxide assay to indirectly measure the activity of the engineered enzyme. Hydrogen peroxide assays are commonly used to quantitate catalytic activity. In accordance with example embodiments, the hydrogen peroxide assay is used to determine catalytic activity of the engineered enzyme. Hydrogen peroxide is a reactive oxygen that is a byproduct of the second enzymatic reaction, in accordance with example embodiments. For example, hydrogen peroxide is a byproduct of the bio-oxidation of the fatty alcohol to produce a fatty aldehyde. By use of the hydrogen peroxide assay to determine the hydrogen peroxide produced in the reaction. In some embodiments, the hydrogen peroxide is determined quantitatively or qualitatively. Examples of suitable hydrogen peroxide assays include colorimetric assays and fluorometric assays. In some embodiments, the hydrogen peroxide assay is performed extracellular. For example, the hydrogen peroxide diffuses across one or more cell membranes to contact the reagents of the assay.

Example embodiments of hydrogen peroxide colorimetric assays uses a hydrogen peroxide reagent that reacts with hydrogen peroxide to produce a color in proportion to the concentration of hydrogen peroxide. In some embodiments, the colorimetric assay includes a catalyst to catalyze a reaction between the hydrogen peroxide reagent and the hydrogen peroxide. Examples of suitable catalysts include horseradish peroxidase and catalase Examples of suitable hydrogen peroxide reagents include 2,2′-azino-bis(3-ethylbenzothiazoline-6-sulfonic acid). An example of a suitable colorimetric assay reacts 2,2′-azino-bis(3-ethylbenzothiazoline-6-sulfonic acid) and hydrogen peroxide in the presence of a catalyst, such as horseradish peroxidase, to produced colored reaction products. The hydrogen peroxide can be determined by monitoring the color change, thus indirectly following the reaction kinetics of the first reaction in block 102. In some embodiments, a spectrophotometer can be used to monitor the color change.

Examples embodiments of hydrogen peroxide fluorometric assays include the use of hydrogen peroxide reagent that reacts with hydrogen peroxide to produce reaction products that are fluorescent. In some embodiments, the fluorometric assay includes a catalyst to catalyze a reaction between the hydrogen peroxide reagent and the hydrogen peroxide. Examples of suitable catalysts include horseradish peroxidase or catalase. Examples of suitable hydrogen peroxide reagents include 10-acetyl-3,7-dihydroxyphenoxazi ne. An example of a fluorometric assay reacts 10-acetyl-3,7-dihydroxyphenoxazine and hydrogen peroxide in the presence of a catalyst, such as horseradish peroxidase, to produce a red-fluorescent oxidation product called resorufin. Light may then be used to trigger fluorescence emissions from the reaction products that can be used to determine the hydrogen peroxide. The specific wavelength of light may be selected, for example, based on the reaction products.

As previously described, the screening method 100 includes a number of different components, including host cells, olefins, and hydrogen peroxide assay components. In some embodiments, these components form a reaction system. In some embodiments, the reaction system further includes a solvent, for example, to which the other components can be added. Examples of suitable solvents include water, alcohols, dimethyl sulfoxide, and acetone. The reactions, for example, in blocks 102, 104, and 106 may be carried out in the solvent. Additional additives may also be included in the reaction system including buffers and surfactants.

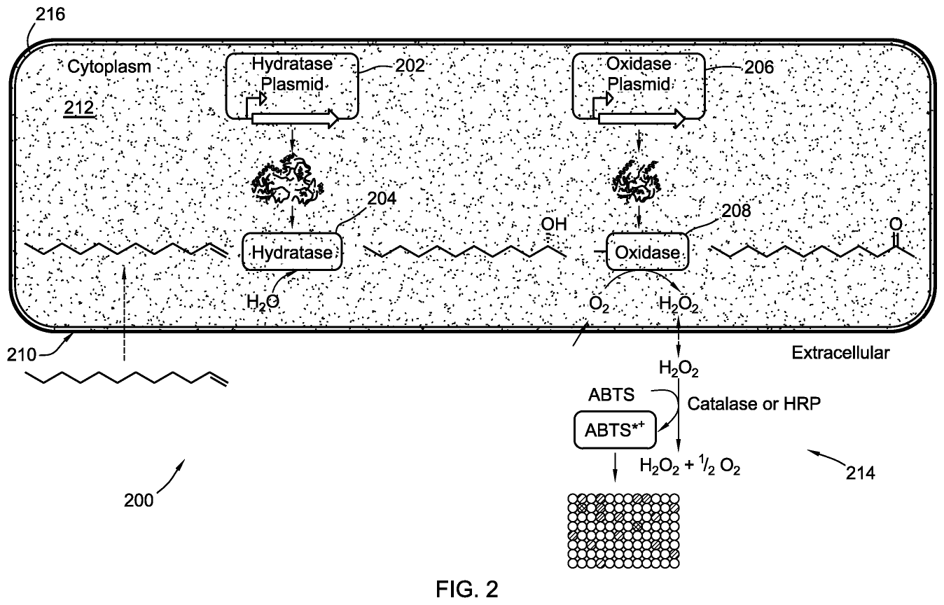

FIG. 2 is a schematic diagram of a screening system 200 for engineered biocatalysts in accordance with one or more embodiments. The screening system 200 can be used, for example, to perform the method 100 shown on FIG. 1 to identify engineered biocatalysts that can oxyfunctionalize olefins with improved performance. In the illustrated embodiment, the screening system 200 includes a recombinant plasmid 202 configured to express an engineered biocatalyst 204 that catalyzes the oxyfunctionalization of olefins. Examples of suitable engineered biocatalyst 204 include engineered microbes and engineered enzymes, such as engineered hydratases (e.g., oleic acid hydratases). In the illustrated embodiment, the screening system 200 further includes a fatty alcohol plasmid 206 configured to express a fatty alcohol oxidase 208 that catalyzes the oxidation of fatty alcohols. As illustrated, the recombinant plasmid 202 and the fatty alcohol plasmid 206 are disposed in a host cell 210. For example, the recombinant plasmid 202 and the fatty alcohol plasmid 204 are disposed in cytoplasm 212 of the host cell 210 as shown on FIG. 2. In the illustrated embodiment, the screening system further includes a hydrogen peroxide assay 214. As illustrated, the hydrogen peroxide assay 214 is extracellular in accordance with one or more embodiments.

In operation, the recombinant plasmid 202 expresses an engineered biocatalyst 204 into the cytoplasm 212 in accordance with one or more embodiments. Example embodiments further include introduction of an olefin into the host cell 210. As illustrated, the olefin can be introduced into the cytoplasm 212 of the host cell 210. In the cytoplasm 212, the olefin reacts with water to form a fatty alcohol. As previously described, this reaction is catalyzed by the engineered biocatalyst 204. The fatty alcohol reacts with oxygen in the presence of a fatty alcohol oxidase 208 that is expressed from the fatty atty alcohol plasmid 206 to form a fatty aldehyde and hydrogen peroxide. In the illustrated embodiment, the hydrogen peroxide diffuses across cell membrane 216 for the hydrogen peroxide assay 214, which can be performed as previously described. In example embodiments of the hydrogen peroxide assay 214, the hydrogen peroxide reacts with a reagent, such as (ABTS), to producer colored reaction products. These reaction products can be observed to determine, for example, the hydrogen peroxide concentration and, thus, indirectly, the activity of the engineered biocatalyst 204.

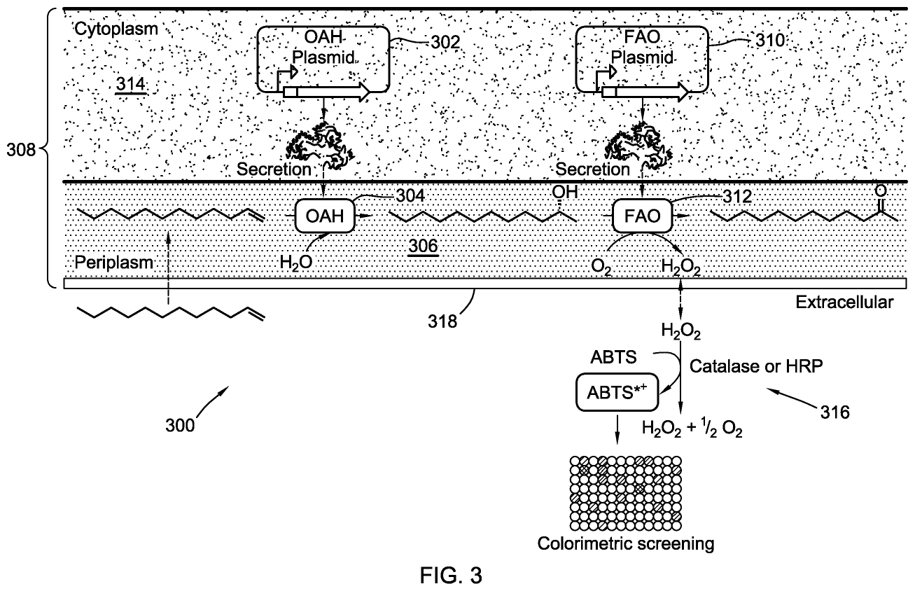

FIG. 3 is a schematic diagram of an alternative screening system 300 for engineered biocatalysts in accordance with one or more embodiments. The alternative screening system 300 can be used, for example, to perform the method 100 shown on FIG. 1 to identify engineered biocatalyst that can oxyfunctionalize olefins with improved performance. In the illustrated embodiment, the screening system 300 includes a recombinant plasmid 302 configured to express an engineered biocatalyst 304 that is secreted into periplasm 306 of the host cell 308 to catalyze the oxyfunctionalization of olefins. Examples of suitable engineered biocatalyst 204 include engineered microbes and engineered enzymes, such as engineered hydratases (e.g., oleic acid hydratases). In the illustrated embodiment, the screening system 300 further includes a fatty alcohol plasmid 310 configured to express a fatty alcohol oxidase 312 that is secreted into the periplasm 306 of the host cell 308 to catalyze the oxidation of fatty alcohols. As illustrated, the recombinant plasmid 302 and the fatty alcohol plasmid 310 are disposed in the host cell 308. For example, the recombinant plasmid 302 and the fatty alcohol plasmid 310 are disposed in cytoplasm 314 of the host cell 308 as shown on FIG. 2. While the recombinant plasmid 302 and fatty alcohol plasmid 310 will secrete their enzymes into the cytoplasm 314, the engineered biocatalyst 304 and the fatty alcohol oxidase 312 are modified with a signal peptide, in accordance with one or more embodiments, so that the enzymes will be secreted into the periplasm 306 upon their expression into the cytoplasm 314. For example, the engineered biocatalyst 304 and the fatty alcohol oxidase 312 can include secretion signal peptides at their N-terminus. In the illustrated embodiment, the screening system further includes a hydrogen peroxide assay 316. As illustrated, the hydrogen peroxide assay 316 is extracellular in accordance with one or more embodiments.

In operation, the recombinant plasmid 314 expresses an engineered biocatalyst 304 that is secreted into the periplasm 306 in accordance with one or more embodiments. Example embodiments further include introduction of an olefin into the host cell 308. As illustrated, the olefin can be introduced into the periplasm 306 of the host cell 308. In the periplasm 306, the olefin reacts with water to form a fatty alcohol. As previously described, this reaction is catalyzed by the engineered enzyme 304. The fatty alcohol reacts with oxygen in the presence of a fatty alcohol oxidase 312 to form a fatty aldehyde and hydrogen peroxide. In some embodiments, the fatty alcohol oxidase 312 is expressed from the fatty atty alcohol plasmid 310 and secreted into the periplasm 306. In the illustrated embodiment, the hydrogen peroxide diffuses across the cell membrane 318 for the hydrogen peroxide assay 316, which can be performed as previously described. In example embodiments of the hydrogen peroxide assay 316 the hydrogen peroxide reacts with a reagent, such as (ABTS), to producer colored reaction products. These reaction products can be observed to determine, for example, the hydrogen peroxide concentration and, thus, indirectly, the activity of the engineered enzyme 304.

### Additional Embodiments

Accordingly, the present disclosure may provide methods and systems for screening biocatalysts to identify engineered biocatalysts that can oxyfunctionalize olefins with improved performance. The methods and systems may include any of the various features disclosed herein, including one or more of the following embodiments.

Embodiment 1. A screening method for identifying engineered biocatalysts, comprising: reacting an olefin with water in the presence of an engineered biocatalyst to produce at least a fatty alcohol having from 4 carbons to 24 carbons; reacting at least a portion of the fatty alcohol with oxygen in the present of a fatty alcohol oxidase to produce a fatty aldehyde and hydrogen peroxide, the fatty aldehyde having from 4 carbons to 24 carbons; and measuring activity of the engineered biocatalyst.

Embodiment 2. The screening method of embodiment 1, wherein the olefin comprises a straight chain α-olefin.

Embodiment 3. The screening method of embodiment 1 or embodiment 2, wherein the engineered biocatalyst comprises an engineered oleic acid hydratase.

Embodiment 4. The screening method of any preceding embodiment, wherein the measuring activity of the engineered biocatalyst comprises determining a concentration of the hydrogen peroxide.

Embodiment 5. The screening method of embodiment 4, wherein the determining the concentration of the hydrogen peroxide is a qualitative determination that indirectly provides an activity of the engineered enzyme.

Embodiment 6. The screening method of embodiment 5, wherein the determining the concentration of the hydrogen peroxide comprises reacting at least a portion of the hydrogen peroxide with a reagent in the presence of a catalyst to produce a colored reaction product.

Embodiment 7. The screening method of embodiment 6, wherein the catalyst comprises horseradish peroxidase and the reagent comprises 2,2′-azino-bis(3-ethylbenzothiazoline-6-sulfonic acid).

Embodiment 8. The screening method of any one of embodiments 4 to 7, wherein the determining the concentration of the hydrogen peroxide comprises reacting at least a portion of the hydrogen peroxide with a reacting to produce one or more fluorescent reaction products.

Embodiment 9. The screening method of embodiment 8, wherein the catalyst comprises horseradish peroxidase and the reagent comprises 10-acetyl-3,7-dihydroxyphenoxazine.

Embodiment 10. The screening method of embodiment 9, wherein the reacting the olefin and reacting the at least the portion of the fatty aldehyde are performed intracellular, and wherein the method further comprises diffusing at least a portion of the hydrogen peroxide across a cell membrane.

Embodiment 11. The screening method of any preceding embodiment, further comprising co-expressing the engineered biocatalyst and the fatty alcohol oxidase into a cytoplasm of a host cell, wherein the reacting the olefin and the reacting at least the portion of the fatty alcohol are performed in the cytoplasm.

Embodiment 12. The screening method of any preceding embodiment, further comprising co-expressing the engineered biocatalyst and the fatty alcohol oxidase into a cytoplasm of a host cell, wherein the engineered biocatalyst and the fatty alcohol oxidase each include a signal peptide such that the engineered biocatalyst and the fatty alcohol oxidase are secreted from the cytoplasm into the periplasm, wherein the reacting the olefin and the reacting at least the portion of the fatty alcohol are performed in the periplasm.

Embodiment 13. The screening method of any preceding embodiment, further comprising repeating the steps of reacting the olefin, reacting at least the portion of the fatty alcohol, and measuring activity of the engineered biocatalyst for a plurality of variants of the engineered biocatalyst.

Embodiment 14. The screening method of any preceding embodiment, wherein the reacting the olefin and reacting at least the fatty alcohol are performed intracellular in a host cell.

Embodiment 15. The screening method of embodiment 14, wherein the host cell comprises Escherichia coli.

Embodiment 16. The screening method of any preceding embodiment, further comprising combing the olefin, a host cell, and components of a hydrogen peroxide assay into a solvent, wherein the host cell contains a recombinant plasmid that expresses the engineered biocatalyst and another plasmid that expresses the fatty alcohol oxidase.

Embodiment 17. A screening system for identifying biocatalysts, comprising: one or more recombinant plasmids configured to express an engineered biocatalyst that oxyfunctionalizes olefins and a fatty alcohol oxidase that oxidizes fatty alcohols; and a hydrogen peroxide assay.

Embodiment 18. The screening system of embodiment 17, wherein the one or more recombinant plasmids are disposed in a cytoplasm of a host cell, and wherein the one or more recombinant plasmids comprise separate plasmids for expressing the engineered biocatalyst and the fatty alcohol oxidase.

Embodiment 19. The screening system of embodiment 17 or embodiment 18, wherein the engineered enzyme and the fatty alcohol oxidase each comprise a signal peptide at their corresponding N-terminus.

Embodiment 20. The screening system of any one of embodiments 17 to 19, wherein the hydrogen peroxide assay comprises a hydrogen peroxide reagent and a catalyst.

To facilitate a better understanding of the present invention, the following examples of certain aspects of some embodiments are given. In no way should the following examples be read to limit, or define, the entire scope of the disclosure.

### EXAMPLES

The following example was performed to evaluate example embodiments of the screening system based on two coupled enzymatic reactions with a hydrogen peroxide assay. A fatty alcohol oxidase (FAO) from Canida tropicalis was cloned and heterogeneously express in E. coli BL21. The FAO was either expressed as the secreted enzyme in the periplasmic space or as the intracellular enzyme. A control was performed with no fatty alcohol added. 2-docecanol was used as the substrate for testing the FAO activity with a colorimetric assay of horseradish peroxidase (HRP) and 2, 2′-azino-bis(3-ethylbenzothiazoline-6-sulfonic acid) (ABTS). The ABTS was provided in dimethyl sulfoxide (DMSO). A tris hydrochloride (tris-HCl) buffer was used to provide pH of 8.

The composition of the reaction system is provided in the table below.

TABLE 1

| | Volume (μL) | Final Concentration | |
|---|---|---|---|
| Tris-HCl (100 mM) | 198 | 50 | mM |
| HRP (2 mg/mL) | 3 | 10 | μg/mL |
| ABTS in DMSO (50 mM) | 30 | 0.25 | mM |

| | | |
|---|---|---|
| 2-Dodecanol | 30 | 0.05 |
| Whole cells with FAO expressed | 160 | N/A |

To initiate the reaction, the ingredients listed above were mixed in a 1.5-mL Eppendorf tube for 5 seconds at room temperature and pressure and then allowed to sit. The reaction was observed at 5 minutes, 10 minutes, and 15 minutes. The results showed that the negative control exhibited clean background for the FAO activity. Specifically, there was no observed color change in the reaction system for the control at any of the observation times. In contrast, the reaction systems with whole cells having the secreted enzyme in the periplasmic and intracellular enzyme both exhibited FAO catalytic activity as the reactions were observed to have increasingly darker shades of blue at the 3 observation times. However, the whole cells with the secreted enzyme in the periplasmic space exhibits faster reaction times with the reaction system turning dark blue much quicker than the reaction system for the whole cells with the intracellular enzyme.

While the disclosure has been described with respect to a number of embodiments and examples, those skilled in the art, having benefit of this disclosure, will appreciate that other embodiments can be devised which do not depart from the scope and spirit of the disclosure as disclosed herein. Although individual embodiments are discussed, the present disclosure covers all combinations of all those embodiments.

While compositions, methods, and processes are described herein in terms of “comprising,” “containing,” “having,” or “including” various components or steps, the compositions and methods can also “consist essentially of” or “consist of” the various components and steps. The phrases, unless otherwise specified, “consists essentially of” and “consisting essentially of” do not exclude the presence of other steps, elements, or materials, whether or not, specifically mentioned in this specification, so long as such steps, elements, or materials, do not affect the basic and novel characteristics of the disclosure, additionally, they do not exclude impurities and variances normally associated with the elements and materials used.

All numerical values within the detailed description are modified by “about” the indicated value, and take into account experimental error and variations that would be expected by a person having ordinary skill in the art.

Many alterations, modifications, and variations will be apparent to those skilled in the art in light of the foregoing description without departing from the spirit or scope of the present disclosure and that when numerical lower limits and numerical upper limits are listed herein, ranges from any lower limit to any upper limit are contemplated.

What is claimed is:

1. A screening method for identifying engineered biocatalysts, comprising:
reacting an olefin with water in the presence of an engineered biocatalyst to produce at least a fatty alcohol having from 4 carbons to 24 carbons;
reacting at least a portion of the fatty alcohol with oxygen in the present of a fatty alcohol oxidase to produce a fatty aldehyde and hydrogen peroxide, the fatty aldehyde having from 4 carbons to 24 carbons; and
measuring activity of the engineered biocatalyst.

2. The screening method of claim 1, wherein the olefin comprises a straight chain α-olefin.

3. The screening method of claim 1, wherein the engineered biocatalyst comprises an engineered oleic acid hydratase.

4. The screening method of claim 1, wherein the measuring activity of the engineered biocatalyst comprises determining a concentration of the hydrogen peroxide.

5. The screening method of claim 4, wherein the determining the concentration of the hydrogen peroxide is a qualitative determination that indirectly provides an activity of the engineered enzyme.

6. The screening method of claim 5, wherein the determining the concentration of the hydrogen peroxide comprises reacting at least a portion of the hydrogen peroxide with a reagent in the presence of a catalyst to produce a colored reaction product.

7. The screening method of claim 6, wherein the catalyst comprises horseradish peroxidase and the reagent comprises 2,2′-azino-bis(3-ethylbenzothiazoline-6-sulfonic acid).

8. The screening method of claim 4, wherein the determining the concentration of the hydrogen peroxide comprises reacting at least a portion of the hydrogen peroxide with a reacting to produce one or more fluorescent reaction products.

9. The screening method of claim 8, wherein the catalyst comprises horseradish peroxidase and the reagent comprises 10-acetyl-3-dihydroxyphenoxazine.

10. The screening method of claim 9, wherein the reacting the olefin and reacting the at least the portion of the fatty aldehyde are performed intracellular, and wherein the method further comprises diffusing at least a portion of the hydrogen peroxide across a cell membrane.

11. The screening method of claim 1, further comprising co-expressing the engineered biocatalyst and the fatty alcohol oxidase into a cytoplasm of a host cell, wherein the reacting the olefin and the reacting at least the portion of the fatty alcohol are performed in the cytoplasm.

12. The screening method of claim 1, further comprising co-expressing the engineered biocatalyst and the fatty alcohol oxidase into a cytoplasm of a host cell, wherein the engineered biocatalyst and the fatty alcohol oxidase each include a signal peptide such that the engineered biocatalyst and the fatty alcohol oxidase are secreted from the cytoplasm into the periplasm, wherein the reacting the olefin and the reacting at least the portion of the fatty alcohol are performed in the periplasm.

13. The screening method of claim 1, further comprising repeating the steps of reacting the olefin, reacting at least the portion of the fatty alcohol, and measuring activity of the engineered biocatalyst for a plurality of variants of the engineered biocatalyst.

14. The screening method of claim 1, wherein the reacting the olefin and reacting at least the fatty alcohol are performed intracellular in a host cell.

15. The screening method of claim 14, wherein the host cell comprises Escherichia coli.

16. The screening method of claim 1, further comprising combing the olefin, a host cell, and components of a hydrogen peroxide assay into a solvent, wherein the host cell contains a recombinant plasmid that expresses the engineered biocatalyst and another plasmid that expresses the fatty alcohol oxidase.

17. A screening system for identifying biocatalysts, comprising:
one or more recombinant plasmids configured to express an engineered biocatalyst that oxyfunctionalizes olefins and a fatty alcohol oxidase that oxidizes fatty alcohols; and
a hydrogen peroxide assay.

18. The screening system of claim 17, wherein the one or more recombinant plasmids are disposed in a cytoplasm of a host cell, and wherein the one or more recombinant plasmids comprise separate plasmids for expressing the engineered biocatalyst and the fatty alcohol oxidase.

19. The screening system of claim 17, wherein the engineered enzyme and the fatty alcohol oxidase each comprise a signal peptide at their corresponding N-terminus.

20. The screening system of claim 17, wherein the hydrogen peroxide assay comprises a hydrogen peroxide reagent and a catalyst.
